(19) United States
(12) Patent Application Publication
(10) Pub. No.: US 2026/0088152 A1
(43) Pub. Date: Mar. 26, 2026

(54) DATA-DRIVEN MENTAL HEALTH PROGRAMMING

(71) Applicant: Charlie Health, Inc, BOZEMAN, MT (US)

(72) Inventors: CAROLINE FENKEL, BOZEMAN, MT (US); JONATHAN KOHLMEIER, BOZEMAN, MT (US); MARK MEGERIAN, BOZEMAN, MT (US)

(73) Assignee: Charlie Health, Inc, BOZEMAN, MT (US)

(21) Appl. No.: 18/893,904

(22) Filed: Sep. 23, 2024

(51) Int. Cl. G16H 20/70; G16H 80/00

(52) U.S. Cl. CPC G16H 20/70; G16H 80/00

(57) ABSTRACT

Methods and systems of data-driven mental health programming is provided. Embodiments ingesting (314) patient data (124) into an enterprise knowledge graph (128), wherein the patient data (124) comprises triples of nodes and relationships representing the data of the patients of the program; deriving (316) a set of patient archetypes (350) from the enterprise knowledge graph (128); assigning (318) a patient (102) to a patient archetype (350) in dependence upon patient attributes (306); assigning (320) the patient (102) to a therapist (386) in dependence upon the patient archetype (350), patient attributes (306), and therapist attributes; and assigning (322) the patient to a treatment modality in dependence upon patient archetypes and patient attributes.

### BACKGROUND

Conventional mental health care systems are limited in how they accept and care for patients. A patient is typically assigned to a therapy program as a referral from a doctor, a therapist, an outpatient program, an emergency room, or other person or program already associated with the patient. Such patients are usually assigned to an in-person clinic for either substance use or mental health. This binary selection of clinics is often because some states have only two general licenses. As such, in-person clinics are not specialized.

Furthermore, regardless of age, diagnosis, modality, or other patient-specific factors, patients are assigned to a clinic and/or group therapy sessions with other individuals based on factors that have little or nothing to do with successful treatment. Such unrelated factors include the patients currently in the clinic or in the group, the patient's location and driving distance from the clinic or group therapy site, insurance type, therapist location and schedule, and so on. Such assignments to clinics and groups for group therapy do not take into consideration the modality of the therapy given, the type of therapist matched with the patient, the age, condition, and experience of other members of a group, and many others in a data driven way.

### BRIEF DESCRIPTION OF THE DRAWINGS

Many aspects of the present disclosure can be better understood with reference to the accompanying drawings. The components in the drawings are not necessarily to scale, with emphasis instead being placed upon illustrating the principles of the disclosure. Moreover, in the drawings, like reference numerals designate corresponding parts throughout the several views.

FIG. 1 sets forth an example system diagram illustrating a system for data-driven mental health programming according to embodiments of the present invention.

FIG. 2 sets forth a line drawing of patient data comprising sets of triples organized as a graph.

FIG. 3 sets forth a flowchart illustrating an example method of data-driven mental health programming according to embodiments of the present invention.

FIG. 4 sets forth a flowchart illustrating an example method of group administration for group therapy according to embodiments of the present invention.

FIG. 5 sets forth a block diagram of an example training phase for data-driven mental health according to embodiments of the present invention.

FIG. 6 sets forth a block diagram of an example implementation phase for data-driven mental health according to embodiments of the present invention.

### DETAILED DESCRIPTION

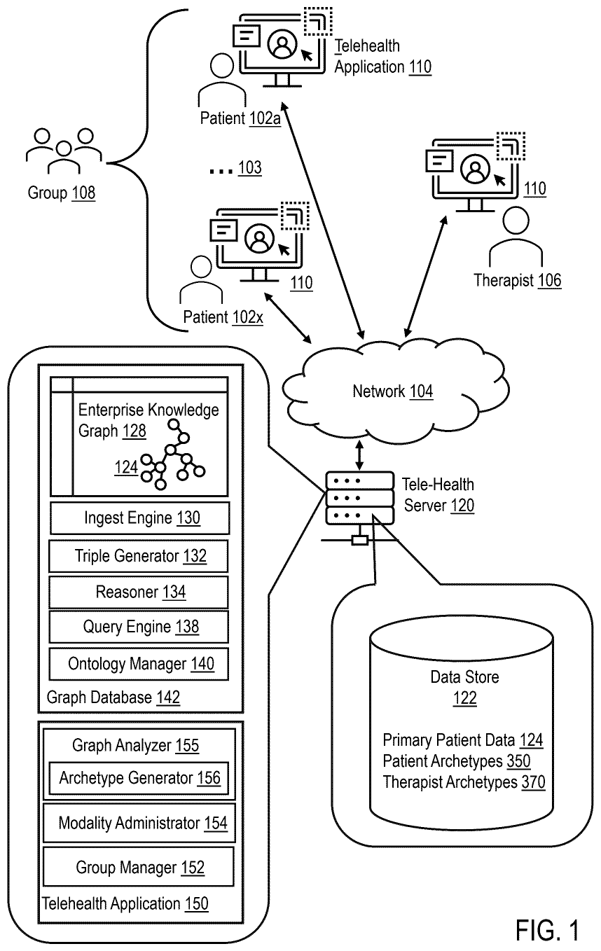

Methods and systems for data-driven mental health programming are described with reference to the attached drawings, beginning with FIG. 1. FIG. 1 sets forth an example system diagram illustrating a system for data-driven mental health programming according to embodiments of the present invention. Programming in this disclosure refers to the structured and organized set of therapeutic activities, interventions, and treatments that are designed to address specific health issues. Telehealth programming refers to the use of digital communication technologies, such as computers, smartphones, and tablets, to provide the healthcare services and manage those structured and organized set of therapeutic activities, interventions, and treatments remotely. Telehealth programs allow patients and healthcare providers to interact without being physically present in the same location, making healthcare more accessible and convenient, especially for those in remote or underserved areas.

The example system of telehealth programming of FIG. 1 is directed to the field of mental health programming although the present invention may be deployed in other areas of telehealth and in-person service delivery as will occur to those of skill in the art. Mental health programming is often guided by individualized treatment plans that outline specific goals, interventions, and expected outcomes for the patient. It may include various therapeutic activities such as individual therapy, group therapy, family therapy, psychoeducation, cognitive-behavioral therapy (CBT), dialectical behavior therapy (DBT), and more. The choice of activities depends on the needs of the individuals involved. Mental health programs often have a structured schedule that often includes multiple sessions per week, sometimes daily, to ensure consistent and continuous care.

Mental health programming can range from lower-intensity outpatient services to more intensive programs like Intensive Outpatient Programs (IOPs) or Partial Hospitalization Programs (PHPs). The level of care is determined by the severity of the mental health condition and the individual's needs. Intensive Outpatient Programming (IOP) refers to a structured treatment program that provides a higher level of care than traditional outpatient therapy but is less intensive than inpatient treatment. IOP is designed for individuals who need more support than what is offered in regular outpatient therapy but do not require 24-hour supervision or care. Many programs include both individual and group therapy sessions. Group sessions offer peer support and a sense of community, while individual sessions allow for personalized attention to specific issues.

The example system of FIG. 1 includes a health server (120) coupled for data communications through a network with several client-side telehealth applications and/or patient monitoring (110). Patients (102a-102x), therapists (106), and other users of the system of FIG. 1 may access therapeutic resources and communicate with one another and automated aspects of the program itself through a client-side telehealth application (110). The telehealth application (110) may be a dedicated client-side application designed to provide resources to the patient (102) and therapist (106), as well as provide video conferencing functionality.

The server (120) of FIG. 1 automated computing machinery, that is, hardware and software, configured to provide resources to patients and therapists, manage modalities, and collect patient data for improved treatment modalities and improved success of patients in the program. The server (120) provides an infrastructure for online resources for the administration and management of the system of FIG. 1. The server (120) provides video conferencing functionality to the patient and the therapist and implements a data-driven approach that utilizes machine learning, advanced graph database technology and the data describing patient outcomes, successful discharges from programs, patient feedback, and other data to tailor modalities and groups for group therapy for patients with an increased likelihood of patient success.

Patient data is collected from the patients of the system of FIG. 1. This patient data (124) is collected through patient intake forms, biopsychosocial assessments, and other methods as will occur to those of skill in the art. A patient intake form for a mental health therapy program is designed to gather comprehensive information about the patient to ensure that the therapist or mental health professional has a clear understanding of the patient's background, needs, and current situation. The form typically includes several sections that cover various aspects of the patient's life and mental health status including personal information, insurance information, and referral information. Intake information also typically includes the reasons for seeking therapy and how the issues are affecting the patient's personal and professional life. Intake may include mental health history including previous therapy, diagnosis, hospitalizations, medications, and behavior and current medical history including medical conditions, allergies, substance use. The intake may include family history including family mental health and medical history as well as other information. The intake form may include daily routine, legal issues, therapy goals, and other information as will occur to those of skill in the art.

Patient data (124) may also include the information gathered in a biopsychosocial assessment. A biopsychosocial assessment is a comprehensive process that provides a multi-dimensional view of the patient's mental health. It is conducted through a combination of interviews, questionnaires, observations, and sometimes input from others, ensuring that the treatment plan is tailored to the individual's unique needs. The assessment is typically carried out with a licensed therapist and includes patient demographics, symptomatology, assessment of depression, anxiety, self-harm, suicidality, family relationships, community relationships and other issues and factors. This assessment helps clinicians gather comprehensive information about the patient's biological, psychological, and social background to better understand the factors contributing to their mental health condition.

The biological assessment typically includes evaluation of the patient's medical history, family history, physical health, and other biological information about the patient. The psychological assessment includes the evaluation of the patient's mental health history, cognitive assessment, emotional state, personality and coping skills, trauma history, and other psychological factors. The social assessment includes evaluation of the patient's social support, living situation, work and education, cultural and spiritual situation, socioeconomic situation, and other information about social factors concerning the patient.

During treatment, data is collected as feedback from patients, survey scores of the group, measurement-based care results throughout treatment, attendance rate, duration of time in program, intensity of the program, therapist feedback, therapist notes and diagnosis, and others. This unprecedented mental health data collected in a single mental health program, with tens of thousands of patients and millions of hours of program therapy is used in the system of FIG. 1 to derive patient archetypes, model successful modalities, and manage successful group therapy for patients to name only a few.

The server of FIG. 1 includes a graph database (142) and a telehealth application and/or monitoring device (150). The example graph database (142) of FIG. 1 is a type of NoSQL database designed to handle and store data structured as graphs. In this context, a graph is a collection of nodes or vertices and relationships or edges that connect pairs of nodes. Nodes represent entities such as people, businesses, accounts, or any other item to be tracked. Edges represent the relationships between nodes. Both nodes and edges can have their own properties. This structure is particularly useful for representing complex relationships and interdependencies between data points, making graph databases a powerful tool for various applications.

The system of FIG. 1 includes an enterprise knowledge graph (128) storing patient data (124). An enterprise knowledge graph (EKG) is a structured, interconnected representation of an organization's data, knowledge, and relationships, designed to enable advanced data integration, retrieval, and analytics across the enterprise. It combines data from various sources within an organization into a unified, semantic framework, enabling better decision-making, data governance, and insight generation.

Enterprise knowledge graphs use semantic technologies, such as ontologies and taxonomies, to define the meaning of data and relationships within the enterprise. This allows for a common understanding of concepts and terms across different departments and systems. EKGs capture not just raw data, but also the context in which that data exists. This includes relationships between entities, the significance of data points, and how different pieces of information are related in real-world scenarios.

Enterprise knowledge graphs support complex queries and analytics that go beyond traditional database capabilities. EKGs are often integrated with machine learning and artificial intelligence (AI) to enhance knowledge discovery, automate processes, and support predictive analytics. The graph structure enables AI models to leverage the rich relationships and context captured in the graph.

The graph database (142) of FIG. 1 includes an ingest engine (130). The ingest engine (130) of FIG. 1 is a system module responsible for importing and processing data into the enterprise knowledge graph (128) of the graph database (128). The ingest engine accurately and efficiently integrates patient data (124) into the graph (128). This integration enables the knowledge graph to provide comprehensive and up-to-date insights, facilitate complex queries, and support advanced analytics across the organization.

The graph database (142) of FIG. 1 includes a triple generator (132). The triple generator (132) of FIG. 1 is an algorithmic tool that automatically creates triples from patient data (124) based on certain rules, patterns, or data inputs. Triples are the basic units of data in a graph model, especially in RDF (Resource Description Framework) databases. A triple is a data structure that consists of three components: a subject, a predicate, and an object. The subject is the entity or resource being described. The predicate is the attribute or relationship of the subject. The object is the value of the attribute or the entity that the subject is related to. For example, in the triple:
Subject: “Joe”
Predicate: “is a”
Object: “patient”
This triple represents the relationship “Joe is a patient.”

The graph database (142) of FIG. 1 includes a reasoner (134). The example reasoner of FIG. 1 derives logical conclusions or new information from existing data in the graph, typically based on a set of rules, ontologies, or logical inference mechanisms. The reasoner applies logical inference rules to the data in the graph. These rules are often based on ontologies, which define the relationships between different types of entities and the properties they can have. The reasoner can use this knowledge to infer new facts that are not explicitly stored in the database but can be logically deduced from the existing data.

The graph database (142) of FIG. 1 includes an ontology manager. The ontology manager of FIG. 1 is a tool for managing ontologies within the database. Ontologies are formal representations of knowledge that define the types, properties, and relationships between entities in a particular domain. They are used to structure and organize data, enabling more sophisticated querying, reasoning, and data integration. The ontology manager allows users to create and define ontologies, editing existing ontologies, ensuring the ontology does not contain logical inconsistencies or violations of constraints and others.

The graph database (142) of FIG. 1 includes a query engine (138). The query engine is responsible for executing queries against the graph data and returning the results. It interprets and processes the queries written in the database's query language, performs the necessary operations to retrieve and manipulate the data, and provides the output in a format requested by the user. The query engine parses the query to ensure it adheres to the syntax and semantics of the query language used by the graph database (e.g., Cypher for Neo4j, GSQL for TigerGraph). It analyzes and optimizes the query to improve performance. This may involve rewriting queries or choosing the most efficient execution plan.

The engine retrieves data from the graph database based on the query's conditions. This involves traversing nodes and edges according to the query's patterns. It matches patterns defined in the query with the graph data. For example, finding all nodes that match a certain label or retrieving nodes connected by specific types of relationships. Examples of query languages and respective engines include Cypher (Neo4j): GSQL (TigerGraph): SPARQL (RDF Databases) and others as will occur to those of skill in the art.

The server (120) of FIG. 1 includes a telehealth server application or patient monitoring system (150) that includes a graph analyzer (155), an analytics layer where machine learning algorithms run on the patient data in the enterprise knowledge graph. This could include traditional graph algorithms as well as more advanced machine learning algorithms designed specifically for graph data, such as Graph Neural Networks (GNNs). The graph analyzer of FIG. 1 implements machine learning models to train models for identifying which patient archetypes are best suited to which forms of therapy and type of treatment, which patient archetypes are best suited to which therapist archetypes, what is the optimal group size for this type of patient and therapy, how the cohesiveness of the group members impacts the outcomes for each patient archetype and treatment modality, when there value in modifying the members of a group, and many others.

The graph analyzer (155) of FIG. 1 includes an archetype generator (156) configured to derive a set of patient archetypes (350) and therapist archetypes (370) from an enterprise knowledge graph (128). An archetype is a standardized, reusable model that represents common patterns or structure of attributes defining a patient, a therapist, or other modeled entity. Archetypes are used to define the fundamental components and relationships that are consistent across different instances or scenarios of patients, therapists, and modalities within the program. The patient archetypes are used to inform group placement, select the modality their archetype best responds to, construct groups, optimize familiarity of group members, match therapists, select the level of care and otherwise tailor treatment programs with increased success.

The archetypes of the present invention are data driven. That is, the particular characteristics of a given archetype are exposed by the data itself. As described in more detail below, archetypes are clinically definable and scoped such that archetypes are useful in managing treatment modalities. Archetypes may be derived using machine learning tools such as clustering. Examples of clustering useful in embodiments of the present invention include k-means clustering, hierarchical clustering, DBSCAN (Density-Based Spatial Clustering of Applications with Noise), Gaussian Mixture Models (GMM), and others as will occur to those of skill in the art.

The application (120) of FIG. 1 includes a modality administrator (154). The modality administrator is configured to use the trained models to assign the patient to an archetype; select one or more therapists in dependence upon the archetype and patient parameters; and assign the patient to a treatment modality. A mental health modality is a therapeutic technique used to treat mental health conditions or to promote psychological well-being. Each modality is based on a particular theoretical framework and is designed to address mental health issues in different ways. The choice of modality often depends on the nature of the mental health condition, the individual's needs, and the goals of the treatment.

The modality administrator (154) of FIG. 1 uses one or more trained models to assign a patient to an archetype in dependence upon patient attributes, information that describe the patient; select a therapist and select one or more modalities for the patient based upon the patient's archetype and attributes. Mental health modalities selected may include cognitive-behavioral therapy, dialectical behavior therapy, psychodynamic therapy, humanistic therapy, mindfulness-based cognitive therapy, acceptance and commitment therapy, interpersonal therapy, family therapy, art therapy, play therapy, somatic therapy, and others as will occur to those of skill in the art.

The application (150) includes a group manager (152). The group manager (152) is configured to use one or more trained models to assign the patient to a group (108) for group therapy in dependence upon the patient archetype, patient attributes, and the patient archetypes, and attributes of other patients in the group. The group manager assigns a patient to a group that has the ideal curriculum, group size, constructed with a mix of patient archetypes that is optimal, and with a therapist that is best equipped for this type of treatment and this group. The group manager (152) of FIG. 1 also assigns patients to groups in dependence upon group cohesion. Cohesion, according to embodiments of the present invention, is a metric representing the degree to which the constituent patients in a group are likely to achieve the common goals of the program. Cohesion leads to the constitute patients in a group having more time with each other and having less turnover in the group.

Data-driven mental health programming in example of FIG. 1 is directed to telehealth programming. This is for explanation and not for limitation. Data-driven mental health programming according to embodiments of the present invention may be suited for in-person mental health programming, telehealth programming, or some combination of both as will occur to those of skill in the art.

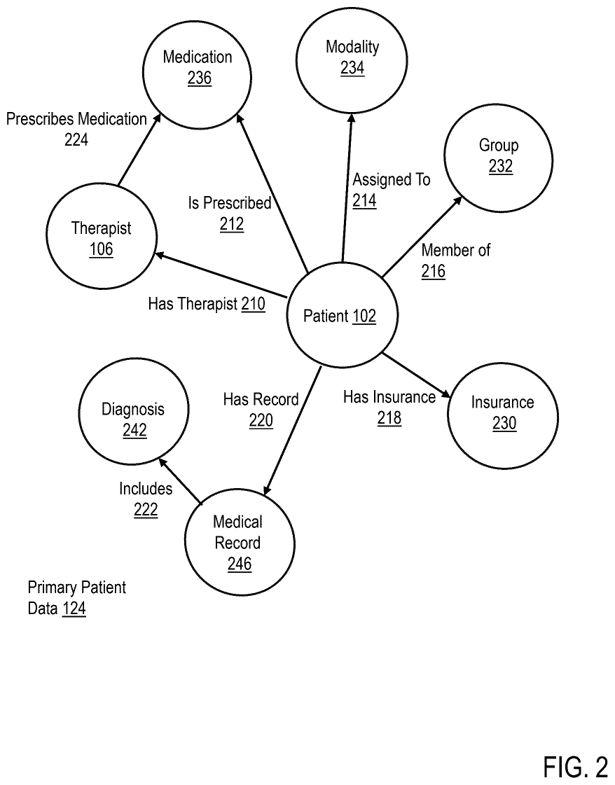

As mentioned above, the system of FIG. 1 includes an enterprise knowledge graph of triples created from patient data. For further explanation, FIG. 2 sets forth a line drawing of a snippet of example patient data (124) comprising sets of triples organized as a graph. The example of FIG. 2 includes 8 nodes representing subjects or objects of triples including a patient (102), a therapist (106), a medication (236), a mental health modality (234), a group (232), an instance of insurance (230), a medical record (246), and a diagnosis (242). The relationships among the nodes form the following example triples: “patient (102) has a (210) therapist (106)”; “patient (102) has (218) insurance (230)”; “patient (102) has a (220) medical record (246)”; “medical record (246) includes (222) diagnosis (242)”; “therapist (106) prescribes (224) medication (238),” “patient (102). Is assigned (214) modality (234)”; “patient (102) is member (215) of group (232).” FIG. 2 illustrates the how graph databases are ideal for applications where relationships are as important as the data itself, such as data-driven mental health programming according to embodiments of the present invention.

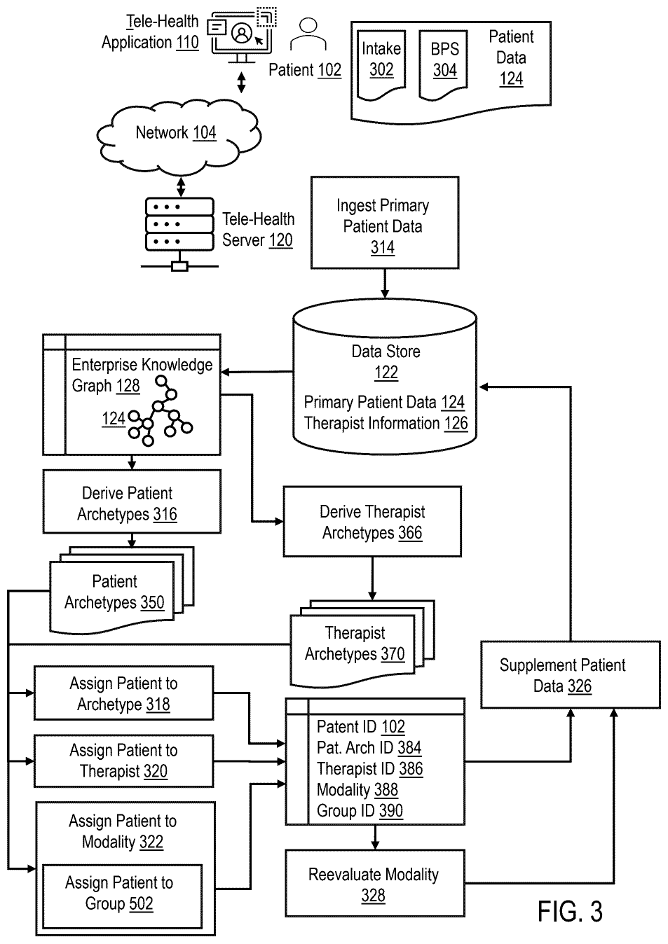

For further explanation, FIG. 3 sets forth a flowchart illustrating a method of data-driven mental health programming. The method of FIG. 3 includes ingesting (314) patient data (124) into an enterprise knowledge graph (128). The patient data (124) of FIG. 1 includes information received from a patient's intake form (302) and a patient's biopsychosocial assessment (304).

The patient data (124) of FIG. 3 includes triples of nodes and relationships representing the data of the patients of the program. Ingesting the patient data into an enterprise knowledge graph may be carried out by extracting data from the native patient data; transforming the data for ingestion; and mapping the nodes and relationships according to the schema of the enterprise knowledge graph. In some embodiments, the patient data is comprised of data of the patients of the program itself without additional patient data from other programs. In alternative embodiments, the patient data may include data derived from patients, therapists, and other data ingested from third party sources.

The method of FIG. 3 includes deriving (316) a set of patient archetypes (350) from the enterprise knowledge graph (128). As mentioned above, a patient archetype is a standardized, reusable model that represents common patterns or structure of attributes defining a patient type.

Deriving (350) a set of patient archetypes from the enterprise knowledge graph (128) may be carried out by dimensionality reduction and clustering. Dimensionality reduction is a process used to reduce the number of input variables or features in a dataset while retaining as much of the essential information as possible. The goal is to simplify the data, making it easier to visualize, process, and analyze, without losing the key patterns or relationships. Common dimensionality reduction techniques include Principal Component Analysis (PCA); t-Distributed Stochastic Neighbor Embedding (t-SNE): Linear Discriminant Analysis (LDA): A technique Autoencoders and others as will occur to those of skill in the art.

Deriving (350) a set of patient archetypes from the enterprise knowledge graph (128) may also include k-means clustering in dependence upon archetype clustering criteria and wherein k is defined as a clinically explainable result. Archetype clustering criteria are considered by the program to be pertinent to the patient's condition for selection of modalities and groups for group therapy. The “k” of the k-means clustering is the number of clusters. K may be selected as a manageable number of archetypes whose derived clusters have centroids and respective clusters sufficiently separated to identify clinically definable archetypes. For example, the patient data may dictate an archetype for adolescents 11-14 that includes high self-harm, low substance abuse, high suicidal ideation, poor family relationships, and particular assessment scores. This archetype may be used to assign patients conforming to that archetype to modalities and groups for group therapy according to embodiment of the present invention.

The use of k-means clustering is for explanation and not for limitation. Examples of other clustering algorithms useful in embodiments of the present invention include hierarchical clustering, DBSCAN (Density-Based Spatial Clustering of Applications with Noise), Gaussian Mixture Models (GMM), and others as will occur to those of skill in the art.

The method of FIG. 3 also includes deriving (366) therapist archetypes (370) in dependence upon patient data (124) and therapist attributes. Therapist archetypes are derived in a manner similar to those of patient archetypes using data describing therapists of the program and in some cases patient data as will occur to those of skill in the art.

The method of FIG. 3 includes assigning (318) a patient (102) to a patient archetype (350) in dependence upon patient attributes (306). Assigning a patient to an archetype in dependence upon patient attributes may be carried out by calculating the cosine similarity of attributes of the patient and a centroid of each patient archetype. Calculating the cosine similarity of the patient and the centroid of each patient archetype includes identifying a cluster whose centroid vector has the smallest angle with the node's vector.

The method of FIG. 3 includes assigning (320) the patient (102) to a therapist (386) in dependence upon the patient archetype (350), patient attributes (306), and therapist attributes. In typical embodiments, the massive amount of patient data accumulated over time is used to train the model to assign the patient to an available therapist most likely to result in a successful outcome for the patient.

The method of FIG. 3 includes assigning (322) the patient to a treatment modality in dependence upon patient archetypes and patient attributes. As with other aspects of the present invention, the patient data accumulated over time is used to train the model to assign the patient to one or more treatment modalities most likely to result in a successful outcome for the patient.

The method of FIG. 3 includes supplementing (326) patient data (124) during the patient's treatment in the program. The patient data in the enterprise knowledge graph is supplemented and augmented throughout the treatment of the patients of the program. Such supplemental and augmented information come in the form of feedback from patients, survey scores of the group, measurement-based care results throughout treatment, attendance rate, duration of time in a particular program, duration of time with an increased care level, such as IOP, summaries of therapist notes, and others as will occur to those of skill in the art.

The method of FIG. 3 includes reevaluating (328) the treatment modality (388) in dependence upon the supplemental patient data. As a patient progresses through a program, the patient's condition may evolve and the treatments available may improve. As such, a treatment modality may be reevaluated during treatment in dependence upon updated patient data, refined archetypes, improved modalities, and other factors as will occur to those of skill in the art.

As mentioned above, group therapy is a key component of many treatment modalities. As such, assigning (322) the patient to a treatment modality in dependence upon patient archetypes and patient attributes according to the method of FIG. 3 includes assigning (502) the patient to a group (390) for group therapy. The patient data accumulated over time is used to train the model to assign the patient to a group for a successful outcome. The trained model is used to assign the patient to a modality in dependence upon the patient archetype, patient attributes, and archetypes and attributes of other patients in the group.

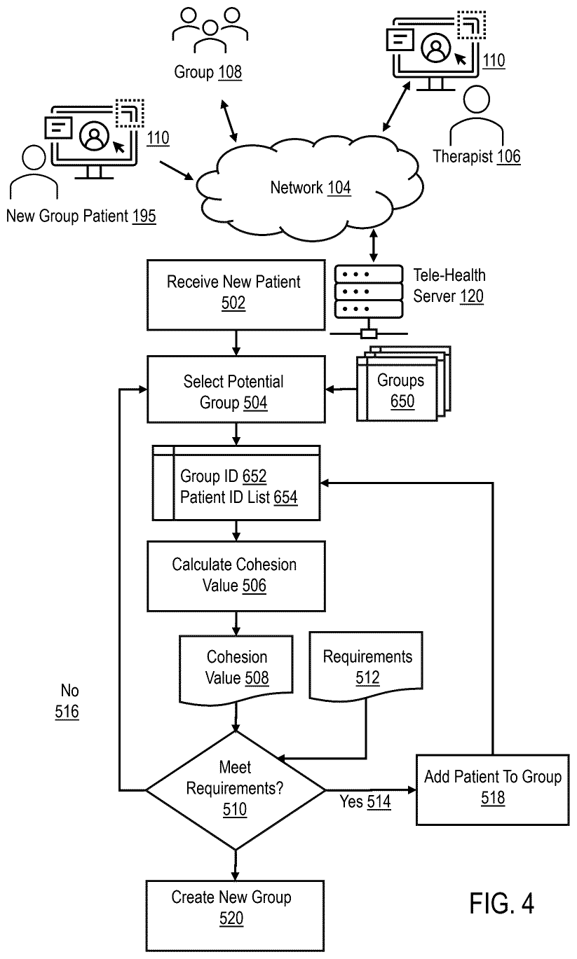

One additional factor found to be useful in assigning a patient to a group for group therapy, is cohesion. Cohesion is a metric representing the degree to which the constituent patients in the group work together effectively to achieve the common goals of the program. For further explanation, FIG. 4 sets forth a flowchart illustrating a method of cohesion-based group administration for group therapy. The method of FIG. 4 includes receiving (502) a new patient (195) for group therapy and selecting (504) a potential group (652) for the new patient (195) in dependence upon patient archetype and patient attributes. Selecting a potential group for the patient includes comparing the patient's archetype and attributes with selection criteria for a potential group including groups that are available addressing the patient's condition, size of available groups, and other factors that will occur to those of skill in the art.

A potential group so selected is just that, a group to which a patient could be assigned. A potential group has the basic requirements for the patient such as allowed archetype for the group, the therapist for the group, the modality of the group, number of members of the potential group and so on as will occur to those of skill in the art.

The method of FIG. 4 also includes calculating (506) a group cohesion value (508) for the potential group (652) including the new patient (195). A cohesion value may be calculated to represent the group cohesion. Such a value may be an alphanumeric value or a multidimensional value allowing complex calculations for cohesion. A group cohesion value may be calculated on number of factors such as time in the group for each patient, a time in group with every other patient in the group; compatibility among of the patient archetypes of patients in the group, therapist archetype of the therapist of the group size of the potential group and other factors as will occur to those of skill in the art.

The method FIG. 4 includes determining (510) whether the calculated group cohesion value (508) meets group cohesion requirements (512). If the calculated group cohesion value meets (514) group cohesion requirements (512), the method of FIG. 4 incudes adding (518) the new group patient (195) to the group (652).

If the calculated group cohesion value does not (516) meet group cohesion requirements, the method of FIG. 4 includes selecting (504) another potential group for the new patient (195). The method of FIG. 4 continues until either a group is selected that meets group cohesion requirements, or no viable group currently exists in the program.

If there is not a potential group for the new patient (195), the method of FIG. 4 includes creating (520) a new group that meets group cohesion requirements (512). Creating a new group may be carried out by populating the new group with the patient and additional members of the program in dependence upon factors such as compatibility among of the patient archetypes of patients in the group and the therapist archetype of the therapist of the group, optimal size for a group, and other factors as will occur to those of skill in art.

Creating a new group may be carried out by populating the new group to meet cohesion requirements and selecting in addition to the patient members of the program based on factors such as time in group for each patient; time in group with every other patient in the group; compatibility among of the patient archetypes of patients in the group; compatibility among of the patient archetypes of patients in the group and the therapist archetype of the therapist of the group, size of the group, and many factors as will occur to those of skill in the art.

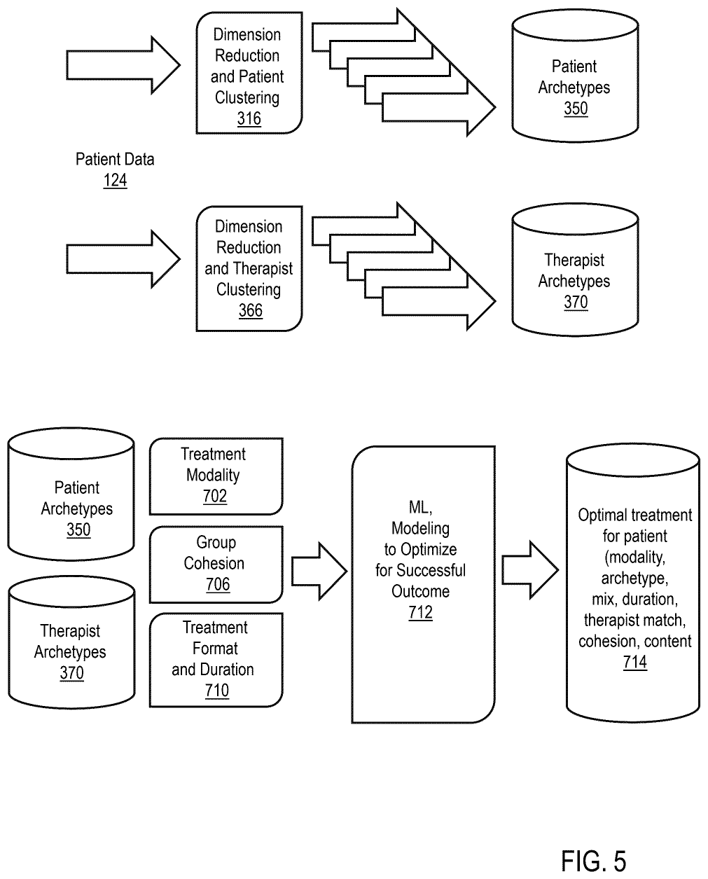

For further explanation, FIG. 5 sets forth a block diagram of an example training phase for data-driven modality according to embodiments of the present invention. The series of models that drive the decisions for optimal treatment planning are trained from patient data (124) accumulated over years. In the example at the outset of the training phase, when data is being accumulated, a mature model with sufficient data does not exist. As more patients complete the program, more data points are collected building an extensive knowledge base of patient factors and how all these factors correlate with the various types of treatment in achieving a successful outcome. In the example of FIG. 5, the patient archetypes (384) and therapist archetypes (386) are derived through dimensionality reduction and patient clustering (316 and 366) of patient data (124).

Having derived the patient archetypes (350) and therapist archetypes (370), models for assigning modality (702), group cohesion (706), and treatment format and duration of modalities (710) are trained (712) with the patient data. Once the initial models have been trained, the trained models (714) may be deployed to for the benefit of the patient.

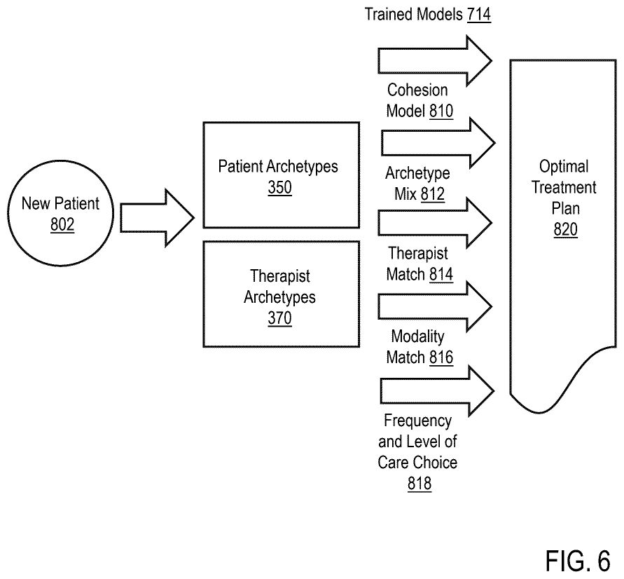

For further explanation, FIG. 6 sets forth a block diagram of an example implementation phase for data-driven modality according to embodiments of the present invention. In the example of FIG. 6, a new patient (802) has the benefit of mature and trained models for patient archetypes (350) and therapist archetypes (370) and mature and trained models for cohesion (810), archetypes and their associated compatibilities (812), optimal therapist match (814), modality assignment (816), and frequency and level of care choices (818). The models develop an optimal treatment plan (820) for the patient (802) that includes modalities and groups with an ideal curriculum, one or more groups of an optimal size and constructed with a mix of patient archetypes that is optimal, and one or more therapists best equipped for the modalities and this group.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A system of data-driven therapeutic programming, the system comprising:
an enterprise knowledge graph including patient data comprising triples of nodes and relationships representing the attributes of patients of the program;
application including:
an archetype generator configured to derive a set of patient archetypes from the enterprise knowledge graph; and
a modality administrator configured to:
assign the patient to an archetype;
select one or more therapists in dependence upon the archetype and patient parameters;
assign the patient to a treatment modality.

2. The system of claim 1 wherein the modality administrator is further configured to supplement the patient data during the patient's treatment in the program and reevaluate the treatment modality in dependence upon the supplemental patient data.

3. The system of claim 1 wherein the archetype generator is further configured to derive therapist archetypes in dependence upon patient data and therapist attributes.

4. The system of claim 3 wherein the modality administrator is further configured to select one or more therapists in further dependence upon therapist archetypes.

5. The system of claim 1 wherein the patient data comprises data exclusively from patients of the program.

6. The system of claim 1 wherein the modality administrator assigns a patient to an archetype in dependence upon patient attributes in dependence upon calculating the cosine similarity of patient and a centroid for each patient archetype.

7. The system of claim 1 further comprising a group manager configured to assign the patient to a group for group therapy in dependence upon the patient archetype, patient attributes, and archetypes and attributes of other patients in the group.

8. The system of claim 6 further comprising a group manager configured to assign the patient to a group for group therapy in dependence upon a group cohesion value.

9. The system of claim 1 further comprising a client application configured for video conferencing.

10. The system of claim 1 wherein the programming comprises telehealth programming.

11. The system of claim 1 wherein the programming comprises intensive outpatient programming.

12. The system of claim 1 wherein the programming includes in-person treatment.

13. A method of data-driven programming, the method comprising:
ingesting patient data into an enterprise knowledge graph, wherein the patient data comprises triples of nodes and relationships representing the data of the patients of the program;
deriving a set of patient archetypes from the enterprise knowledge graph;
assigning a patient to a patient archetype in dependence upon patient attributes;
assigning the patient to a therapist in dependence upon the patient archetype, patient attributes, and therapist attributes; and
assigning the patient to a treatment modality in dependence upon patient archetypes and patient attributes.

14. The method of claim 13 further comprising:
supplementing patient data during the patient's treatment in the program; and
reevaluating the treatment modality in dependence upon the supplemental patient data.

15. The method of claim 13 further comprising deriving therapist archetypes in dependence upon patient data and therapist attributes.

16. The method of claim 13 wherein the patient data comprises data exclusively from patients of the program.

17. The method of claim 13 further comprising dimensionality reduction of the patient data.

18. The method of claim 13 wherein deriving a set of patient archetypes from the enterprise knowledge graph further comprises k-means clustering the nodes of the enterprise knowledge graph in dependence upon archetype clustering criteria and wherein the output is defined as a clinically explainable result.

19. The method of claim 13 wherein assigning a patient to an archetype in dependence upon patient attributes further comprises calculating the cosine similarity of patient and a centroid of each patient archetype.

20. The method of claim 13 wherein ingesting the patient data into an enterprise knowledge graph comprises:
extracting data from the native patient data;
transforming the data for ingestion; and
mapping the nodes and relationships according to the schema of the enterprise knowledge graph.

21. The method of claim 13 wherein assigning the patient to a treatment modality in dependence upon patient archetypes and patient attributes further comprises assigning the patient to a group for group therapy in dependence upon the patient archetype, patient attributes, and archetypes and attributes of other patients in the group.

22. The method of claim 21 wherein assigning the patient to a group for group therapy is carried out in further dependence upon a group cohesion value calculated for a potential group to include the patient.

23. The method of claim 13 wherein the programming comprises group therapy.

24. A method of cohesion-based group administration for group therapy, the method comprising:
receiving a new patient for group therapy;
selecting a potential group for the new patient in dependence upon patient archetype and patient attributes;
calculating a group cohesion value for the potential group including the new patient; and
determining whether the calculated group cohesion value meets group cohesion requirements;
if the calculated group cohesion value meets inclusion group cohesion requirements, adding the new group patient to the group; and
if the calculated group cohesion value does not meet group cohesion requirements, selecting another potential group for the new patient; and
If there is not another potential group for the new patient, creating a new group that meets group cohesion requirements.

25. The method of claim 24 wherein calculating a group cohesion value for the potential group includes determining a time in group for each patient.

26. The method of claim 24 wherein calculating a group cohesion value for the potential group includes determining, for each patient in the group, a time in group with every other patient in the group.

27. The method of claim 24 wherein calculating a group cohesion value for the potential group includes evaluating compatibility among of the patient archetypes of patients in the group.

28. The method of claim 24 wherein calculating a group cohesion value for the potential group includes evaluating compatibility among of the patient archetypes of patients in the group and the therapist archetype of the therapist of the group.

29. The method of claim 24 wherein calculating a group cohesion value for the potential group includes assessing the size of the potential group.

30. A system of group administration for group therapy, the system comprising automated computing machinery configured for:
receiving a new patient for group therapy;
selecting a potential group for the new patient in dependence upon patient archetype and patient attributes;
calculating a group cohesion value for the potential group including the new patient; and
determining whether the calculated group cohesion value meets group cohesion requirements;
if the calculated group cohesion value meets inclusion group cohesion requirements, adding the new group patient to the group; and
if the calculated group cohesion value does not meet group cohesion requirements, selecting another potential group for the new patient; and
If there is not another potential group for the new patient, creating a new group that meets group cohesion requirements.

31. The system of claim 30 wherein calculating a group cohesion value for the potential group includes determining a time in group for each patient.

32. The system of claim 30 wherein calculating a group cohesion value for the potential group includes determining, for each patient in the group, a time in group with every other patient in the group.

33. The system of claim 30 wherein calculating a group cohesion value for the potential group includes evaluating compatibility among of the patient archetypes of patients in the group.

34. The system of claim 30 wherein calculating a group cohesion value for the potential group includes evaluating compatibility among of the patient archetypes of patients in the group and the therapist archetype of the therapist of the group.

35. The system of claim 30 wherein calculating a group cohesion value for the potential group includes assessing the size of the potential group.
